(19) United States
(12) Patent Application Publication
(10) Pub. No.: US 2009/0203671 A1
(43) Pub. Date: Aug. 13, 2009

(54) METHOD OF TREATING CANCER

(75) Inventors: Keith B. Glaser, Lake Villa, IL (US); Omar Jameel Shah, Lake Villa, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/277,382

(22) Filed: Nov. 25, 2008

Related U.S. Application Data
(60) Provisional application No. 60/990,436, filed on Nov. 27, 2007.

(51) Int. Cl. A61K 31/55

(52) U.S. Cl. 514/215

(57) ABSTRACT

Methods of treating cancer in patients comprising administering thereto aurora kinase inhibitors and TRAIL (tumor necrosis factor (TNF)-related apoptosis inducing ligand) inhibitors is disclosed.

This application claims priority to U.S. provisional Application Ser. No. 60/990,436, filed Nov. 27, 2007.

### FIELD OF THE INVENTION

This invention pertains to methods of treating cancer in patients comprising administering thereto aurora kinase inhibitors and TRAIL (tumor necrosis factor (TNF)-related apoptosis inducing ligand) inhibitors.

### BACKGROUND OF THE INVENTION

Small molecule inhibitors of the Aurora-A and -B kinases interfere with mitotic centrosome function and disrupt the mitotic spindle assembly checkpoint resulting in polyploidization and apoptosis of proliferating cells. As such, aurora kinase inhibitors are at various stages of clinical development as anticancer agents. A separate method of treatment is direct induction of cell death by activation of death receptor-mediated apoptosis. TRAIL, a recently identified member of the growing TNF superfamily, binds to its cognate “death” receptors DR4 and DR5 as well as decoy receptors DcR1 and DcR2.

### BRIEF DESCRIPTION OF THE FIGURES

FIG. 1 shows the synergistic anti-proliferative effects of combining Aurora B inhibitors, AZD 1152-HQPA, VX-680/MK0457 or MLN-8054 (at 1 μM), with TRAIL in the D54MG glioblastoma multiforme cell line.

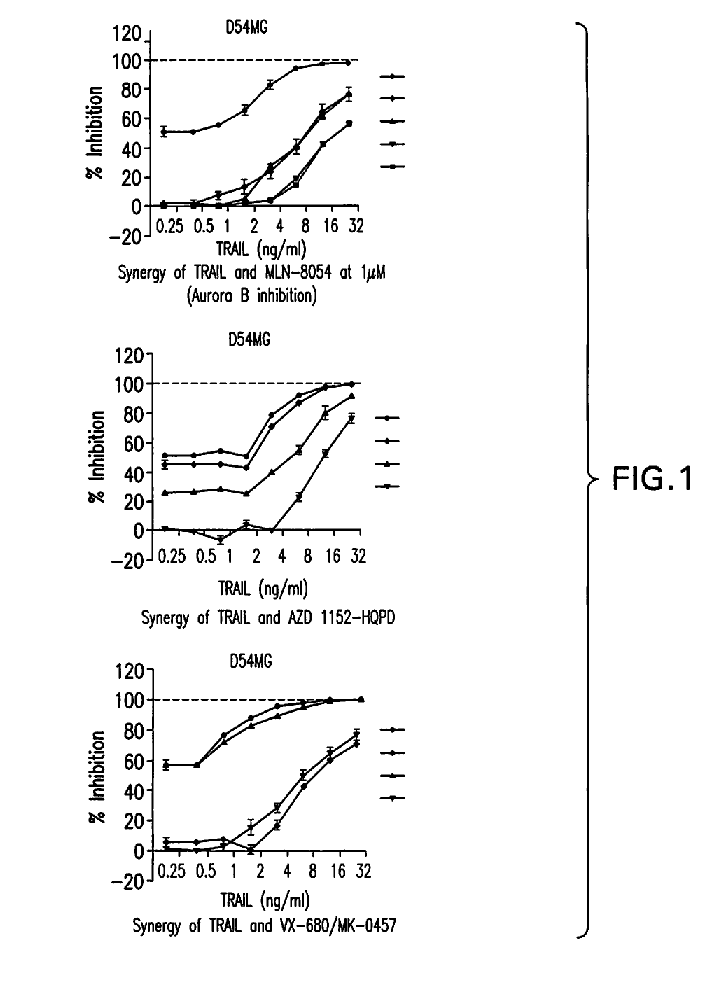

FIG. 2 shows synergistic anti-proliferative effects of Aurora B inhibitors in A172 and LN18 glioblastoma cell lines. Synergy was not observed in glioblastoma cell lines that overexpress the TRAIL decoy receptor (non-functional) osteoprotegerin, OPG.

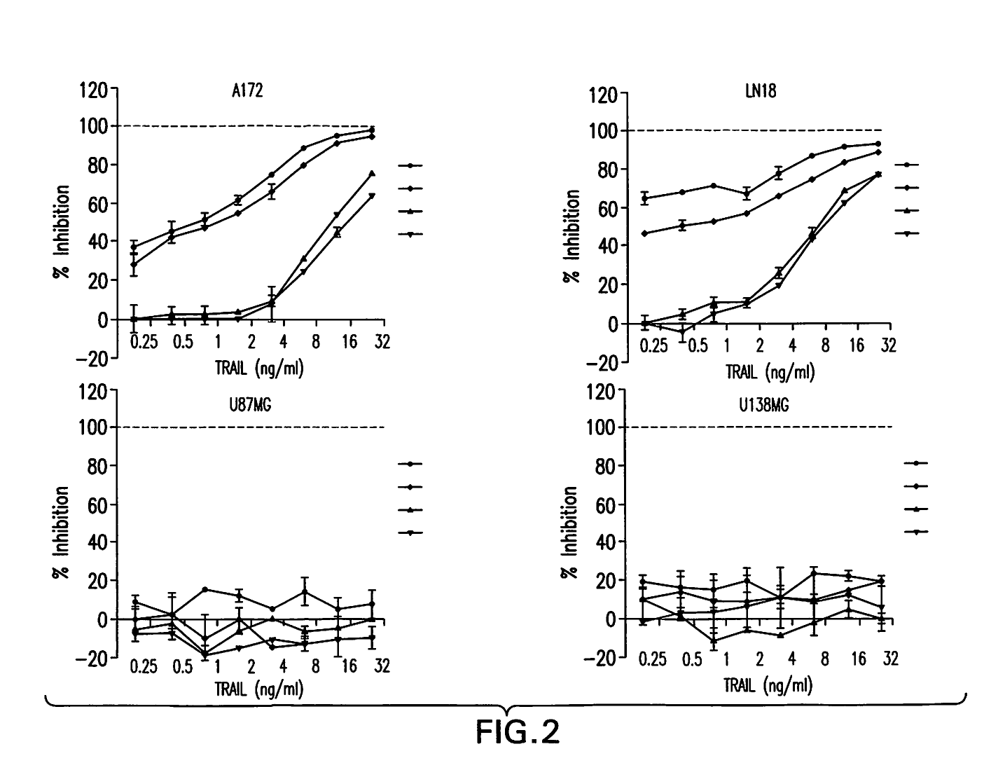

### SUMMARY OF THE INVENTION

One embodiment of this invention pertains to methods of treating cancer in a mammal comprising administering thereto a therapeutic amount of an aurora kinase inhibitor and a therapeutic amount of a TRAIL inhibitor, wherein the aurora kinase inhibitor and the TRAIL inhibitor demonstrate a greater than additive therapeutic effect.

Another embodiment pertains to methods of treating glioblastoma in a mammal comprising administering thereto a therapeutic amount of an aurora kinase inhibitor and a therapeutic amount of a TRAIL inhibitor, wherein the aurora kinase inhibitor and the TRAIL inhibitor demonstrate a greater than additive therapeutic effect.

Still another embodiment pertains to methods of treating glioblastoma in a human comprising administering thereto a therapeutic amount of an aurora kinase inhibitor and a therapeutic amount of a TRAIL inhibitor, wherein the aurora kinase inhibitor and the TRAIL inhibitor demonstrate a greater than additive therapeutic effect.

Still another embodiment of this invention pertains to methods of treating cancer in a mammal comprising administering thereto a therapeutic amount of AZD 1152-HQPA and a therapeutic amount of a TRAIL inhibitor, wherein the aurora kinase inhibitor and the TRAIL inhibitor demonstrate a greater than additive therapeutic effect.

Still another embodiment pertains to methods of treating glioblastoma in a mammal comprising administering thereto a therapeutic amount of AZD 1152-HQPA and a therapeutic amount of a TRAIL inhibitor, wherein the aurora kinase inhibitor and the TRAIL inhibitor demonstrate a greater than additive therapeutic effect.

Still another embodiment pertains to methods of treating glioblastoma in a human comprising administering thereto a therapeutic amount of AZD 1152-HQPA and a therapeutic amount of a TRAIL inhibitor, wherein the aurora kinase inhibitor and the TRAIL inhibitor demonstrate a greater than additive therapeutic effect.

Still another embodiment of this invention pertains to methods of treating cancer in a mammal comprising administering thereto a therapeutic amount of VX-680/MK0457 and a therapeutic amount of a TRAIL inhibitor, wherein the aurora kinase inhibitor and the TRAIL inhibitor demonstrate a greater than additive therapeutic effect.

Still another embodiment pertains to methods of treating glioblastoma in a mammal comprising administering thereto a therapeutic amount of VX-680/MK0457 and a therapeutic amount of a TRAIL inhibitor, wherein the aurora kinase inhibitor and the TRAIL inhibitor demonstrate a greater than additive therapeutic effect.

Still another embodiment pertains to methods of treating glioblastoma in a human comprising administering thereto a therapeutic amount of VX-680/MK0457 and a therapeutic amount of a TRAIL inhibitor, wherein the aurora kinase inhibitor and the TRAIL inhibitor demonstrate a greater than additive therapeutic effect.

Still another embodiment of this invention pertains to methods of treating cancer in a mammal comprising administering thereto a therapeutic amount of MLN-8054 and a therapeutic amount of a TRAIL inhibitor, wherein the aurora kinase inhibitor and the TRAIL inhibitor demonstrate a greater than additive therapeutic effect.

Still another embodiment pertains to methods of treating glioblastoma in a mammal comprising administering thereto a therapeutic amount of MLN-8054 and a therapeutic amount of a TRAIL inhibitor, wherein the aurora kinase inhibitor and the TRAIL inhibitor demonstrate a greater than additive therapeutic effect.

Still another embodiment pertains to methods of treating glioblastoma in a human comprising administering thereto a therapeutic amount of MLN-8054 and a therapeutic amount of a TRAIL inhibitor, wherein the aurora kinase inhibitor and the TRAIL inhibitor demonstrate a greater than additive therapeutic effect.

### DETAILED DESCRIPTION OF THE INVENTION

To identify candidate apoptosis-sensitizing genes that could be exploited in combination with Aurora kinase inhibitors in malignant glioma, global gene expression analysis in a D54MG glioma cell derivative treated with three Aurora kinases inhibitors chosen for their distinctive selectivities was carried out: MLN8054 (4-((9-chloro-7-(2,6-difluorophenyl)-5H-pyrimidol[5,4-d][2]benzazepin-2-yl)amino)benzoic acid, Aurora-A-selective), AZD1152-HQPA (Aurora-B-selective), and VX-680 (Aurora-A/B). This analysis identified the TRAIL death receptor, DR5, as an apoptosis sensitizing gene induced selectively following inhibition of Aurora-B kinase cancer cells. In glioma cell lines where DR5 was induced following polyploidization, the sensitivity, kinetics, and magnitude of TRAIL-mediated apoptosis were enhanced. These data shed light on the apoptotic program induced during polyploidization and suggest that the combination of TRAIL and inhibitors of Aurora B kinase can selectively enhance TRAIL-induced apoptosis and anti-proliferative effects through the up-regulation of DR5, the TRAIL death receptor.

Compounds of this invention may be administered, for example, bucally, ophthalmically orally, osmotically, parenterally (intramuscularly, interparenterally, intrasternally, intravenously, subcutaneously), rectally, topically, transdermally and vaginally.

Therapeutically effective amounts of a compound of this invention depend on recipient of treatment, disease treated and severity thereof, composition comprising it, time of administration, route of administration, duration of treatment, potency, rate of clearance and whether or not another drug is co-administered. The amount of a compound of this invention used to make a composition to be administered daily to a patient in a single dose or in divided doses is from about 0.001 to about 200 mg/kg body weight. Single dose compositions contain these amounts or a combination of submultiples thereof.

Compounds of this invention may be administered with or without an excipient. Excipients include, for example, encapsulators and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof.

Excipients for preparation of compositions comprising a compound of this invention to be administered orally include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethylcellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water and mixtures thereof. Excipients for preparation of compositions comprising a compound of this invention to be administered ophthalmically or orally include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water and mixtures thereof. Excipients for preparation of compositions comprising a compound of this invention to be administered osmotically include, for example, chlorofluoro-hydrocarbons, ethanol, water and mixtures thereof. Excipients for preparation of compositions comprising a compound of this invention to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof. Excipients for preparation of compositions comprising a compound of this invention to be administered rectally or vaginally include, for example, cocoa butter, polyethylene glycol, wax and mixtures thereof.

### Cell Proliferation Assay

Cells were seeded into 96-well plates at 2,500 cells per well and allowed to adhere overnight. Compounds and TRAIL were added the following day, and cells were incubated at 37° C. for 72 h. Inhibition of cell proliferation was determined using CellTiter-Glo Luminescence Cell Viability Assay (Promega, cat# G7570) as suggested by the manufacturer. Percent inhibition of viability was determined relative to cells treated with DMSO alone (0.1%). Data are representative of at least three independent experiments with each data point carried out in triplicate.

### Western Blot Analysis of DR5

After treatment of cells with appropriate Aurora B inhibitors, cells were lysed in modified RIPA buffer containing protease inhibitors. The cell lysate was electrophoresed using NuPAGE Bis-Tris 4-12% gels, transferred to Immobilon-FL membranes (Millipore), and probed for DR5 (gene: TNFRSF10B) using a Cell Signaling Technologies rabbit polyclonal antibody. Visualization of DR5 was performed on a LiCor infrared imaging system using an anti-rabbit IgG Alexa-680 labeled antibody.

The foregoing is meant to illustrate the invention but not to limit it. Variations and changes obvious to one skilled in the art are intended to be within the scope of the invention as defined in the appended claims.

We claim:

1. A method of treating cancer in a mammal comprising administering thereto a therapeutic amount of an aurora kinase inhibitor and a therapeutic amount of a TRAIL inhibitor, wherein the aurora kinase inhibitor and the TRAIL inhibitor demonstrate a greater than additive therapeutic effect.

2. The method of claim 1, wherein the cancer is glioblastoma.

3. A method of treating glioblastoma in a mammal comprising administering thereto a therapeutic amount of an aurora kinase inhibitor and a therapeutic amount of a TRAIL inhibitor, wherein the aurora kinase inhibitor and the TRAIL inhibitor demonstrate a greater than additive therapeutic effect.
